(19) United States
(12) Patent Application Publication
(10) Pub. No.: US 2002/0187468 A1
(43) Pub. Date: Dec. 12, 2002

(54) METHOD AND DEVICE FOR FIXING MICRO-AND/OR NANO-OBJECTS

(76) Inventors: WINFRIED JENTSCH, BERLIN (DE); ULRICH SCHMUCKER, IRXLEBEN (DE); MIKHAIL ZUBTSOV, LUCKENWALDE (DE)

Correspondence Address: BIRCH STEWART KOLASCH & BIRCH, PO BOX 747, FALLS CHURCH, VA, 22040-0747, US

(21) Appl. No.: 09/463,136

(22) PCT Filed: May 20, 1999
(86) PCT No.: PCT/EP99/03476

(30) Foreign Application Priority Data
May 20, 1998 (DE) 198 23 660.3

(51) Int. Cl. C12Q 1/68; C12P 19/34

(52) U.S. Cl. 435/6; 435/91.2

(57) ABSTRACT

The invention relates to a method for obtaining recombinant HbsAg. According to said method, recombinant methylotrophic yeast cells that are capable of expressing HbsAg are broken down using a high-pressure homogenizer and the HBsAg is extracted from the resulting cell debris. The inventive method is characterised by a high product yield per g cell dry weight and is therefore a considerable improvement on known methods for obtaining HBsAg from micro-organisms.

### DESCRIPTION

The invention relates to a method and a device for fixing micro- and/or nano-objects with the characteristics of the species as named in the generic part of claims 1 and 15.

For the execution of complex biochemical analysis, such as DNA-, virus- or gene-analysis, the analysis and interpretation of a great number of single reactions is necessary. The state of the art is the parallel execution of few 10 . . . 100 analysis in so called microtitre plates. Therefore, the to be examined substance, which is placed on plates with regularly arranged depressions, is brought to a reaction with different analysis substances. The introduction of the test- and analysis-substances can take place fully automatically with so called pipetting robots, wherein amounts of substances of few 10 . . . 100 micro-liters are used. This method and the following extensive processing steps for the dissolving-out and the interpretation of the desired chemical reactions necessitate a very high equipment and time effort, so that such analysis only can be performed in special laboratories.

According to a method of U.S. Pat. No. 5,445,934 a miniaturisation and simultaneous carrying out of the analysis is achieved because any nucleotide chains (oligo-nucleotides) can be synthesised on a support-chip by utilization of the four nucleotide basic elements and of masking technology known from the semi-conductor-technology. In this way a couple million different oligo-nucleotides can be produced on a chip and can be interpreted depending on the reaction with the test-substances by means of known methods (e.g. fluorescence analysis). The advantage of the high simultaneity is opposed by very small flexibility, as for each new to be detected substance (e.g. gene or gene portion) a new masking set with corresponding high costs has to be produced.

A further known method of the bio-chemical analytical chemistry uses balls made from glass, metal or plastics with a diameter of few micrometers up to few hundreds micrometers as a support for the analysis substances. With this for example oligo-nucleotides are directly or through so called linkers set on the balls. This method is especially used for in-vivo-analysis, in which these balls are injected into a watery solution directly in the cells, vessels, etc.

According to EP 0 040 943 B1 holes are made in the support, into which cage-like holding devices made from wire or similar are hung. Several balls are then positioned and fixed in these cages in a manner described not in detail.

The production of such structures should be extremely work intensive. The realisation is not known. The miniaturisation is here limited. Furthermore, such a structure would be mechanically very instable and therefore would hardly be of practical use. The positioning and fixing of the balls have not been solved.

The invention is based on the object to achieve a simple, cheap and for a mass-production suitable method including a corresponding device, which enable an exact and reproducible positioning and fixing of a large number of bio-chemical active micro- and/or nano-objects in the form of three-dimensional shaped bodies such as micro-balls and macro-molecules on a common support.

The solution according to the invention is characterised in that the number of shaped bodies and therefore the to be analysed substances can be easily adapted to the requirements of the analysis to be carried out. This means that advantageously few to several ten-thousand substances can be determined. Furthermore, the arrangement of the coating of the shaped bodies can as regards to the chemical composition as well as the positioning on the support very easily be adapted to the requirements. Shaped bodies with the same coating can especially be provided several times on the support. Because of this redundancy an increase in the evaluation accuracy can be achieved. Therefore the method of analysis becomes very flexible and can be easily miniaturised (e.g. several ten-thousand balls per square-centimetre). Furthermore the coating of a ball consists of fractional part of a pico-litre of the analysis substance. Therefore, the consumption of partly very expensive analysis substances is reduced by several orders of magnitude compared with the microtitre method.

As shaped bodies according to the invention, known ball-like objects as well as macro-molecules can be used, which are coated with a specific analysis substance and which are dispersed in a watery, buffered solution. They are put into a capillary tube, preferably made from glass, which at its upper end has a filling hole having an inner diameter, which makes a filling process with a normal pipette or a pipetting robots possible. The capillary tube is tapered downwards to an outlet opening, so that it has at the bottom portion an inner diameter extending over a defined length, which is larger than the ball diameter, but smaller than twice the ball diameter. With a sufficient small capillary diameter the capillary force and the adhesion force prevent an exiting of the liquid and therefore the exiting of the balls from the outlet opening. By applying a force on the liquid phase in the capillary tube—e.g. by applying a pressure difference between the upper capillary filling hole and the lower capillary outlet opening (either an excess pressure at the top or a vacuum at the bottom) by means of electrostatical, magnetical or other physical forces- an exiting of the liquid phase, which contains the shaped bodies dispersed, takes place at the bottom end of the capillary tube.

According to the invention several of such capillary tubes, which are filled with shaped bodies having different coatings and characteristics, are regularly arranged to a positioning head, preferably in a hexagonal or in a rectangular pattern, so that at least the outlet openings and also the filling holes are arranged in a plane vertical to the capillary axis. This plane is following designated as the outlet plane.

If a support is placed parallel below the outlet plane at a distance, which is smaller than the diameter of the shaped body, and if the mentioned pressure difference is applied, the liquid phase as well as a single ball will exit each capillary onto the support, if the shaped body is a ball. The support can here be plane or structured.

The exiting balls have to be fixed on the support before the positioning head and the support, after finishing the positioning process, again are separated from each other, as otherwise the surface tension can draw back the balls into the capillaries when tearing the liquid film.

The fixing of the exited and placed balls can take place in different ways. For example the use of balls with magnetic core and the placing of a magnetic field, as well as the use of an electrostatic load is possible. It is of advantage to produce directly a permanent fixing. This is achieved according to the invention in such a way, that the support is coated with a suitable substance before the positioning of the balls or that the support directly consists of this substance, which enters into a chemical bonding with the balls, their coating or parts thereof For example, a pre-polymer able to be photopolymerised or a cross-linker can be used as a coating, which makes the fixing of the shaped bodies under the influence of the TV-lamp possible.

The exited liquid can be removed by different known methods, like evaporation, via drainage elements in the support or even by using additional capillaries for sucking off the liquid. A part of the liquid withdraws directly back into the capillaries because of the surface tension while withdrawing the positioning head. This effect can be increased in such a way that the material coupling, buffer liquid—support coating, is selected in such a way, that no wetting takes place.

After the fixing the positioning head and the support are separated from each other by suitable actuators. After this the next positioning process can take place.

During the movement of the balls in the capillaries it may happen, that these form clusters (agglutinate) because of the coagulation and/or adhesion effects, what would make the positioning impossible.

According to the invention this problem is solved in the way that the balls are electrostatically charged in the same sense—either by applying an exterior electrical field or preferably by modifying the coating with polar groups of the same polarity-. In this case the process of the “pressing-out” of the ball out off the outlet opening can very effectively be supported in such a way that a charge with opposing polarity is applied to the support.

After finishing the positioning and fixing process the balls are covered with a suitable gel to prevent a complete drying out, what can lead to a bio-chemical degradation of the analysis substances. Finally follows a covering with a mechanical protection layer, e.g. a film. This completes the production of the analysis chips.

The invention will be described exemplary in detail with reference to the accompanying drawings.

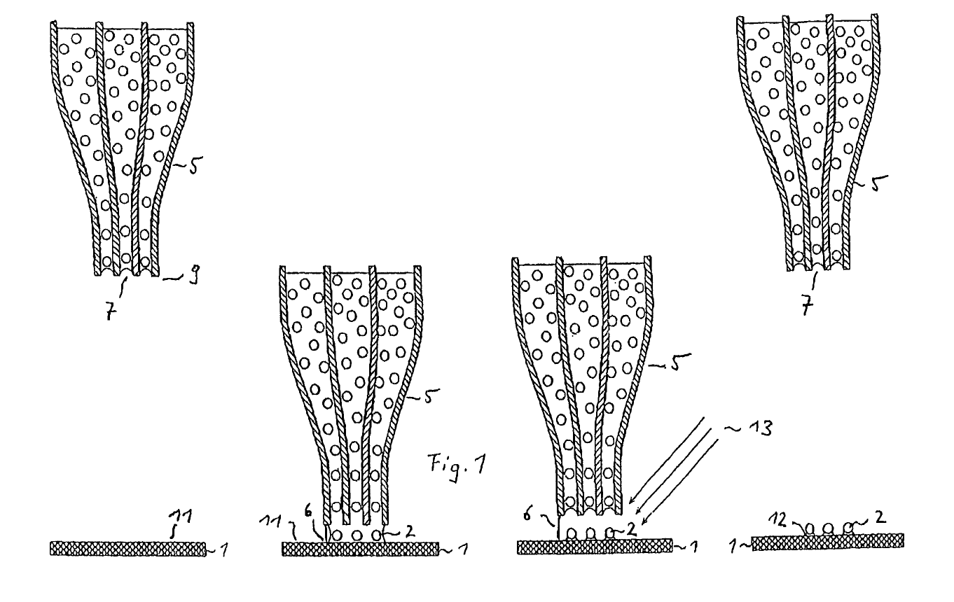

FIG. 1 is a schematic step-like view of a positioning and fixing process,

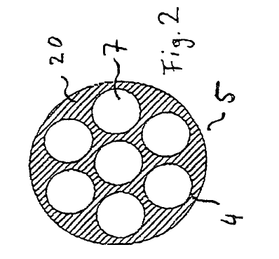

FIG. 2 is a top view of the outlet plane,

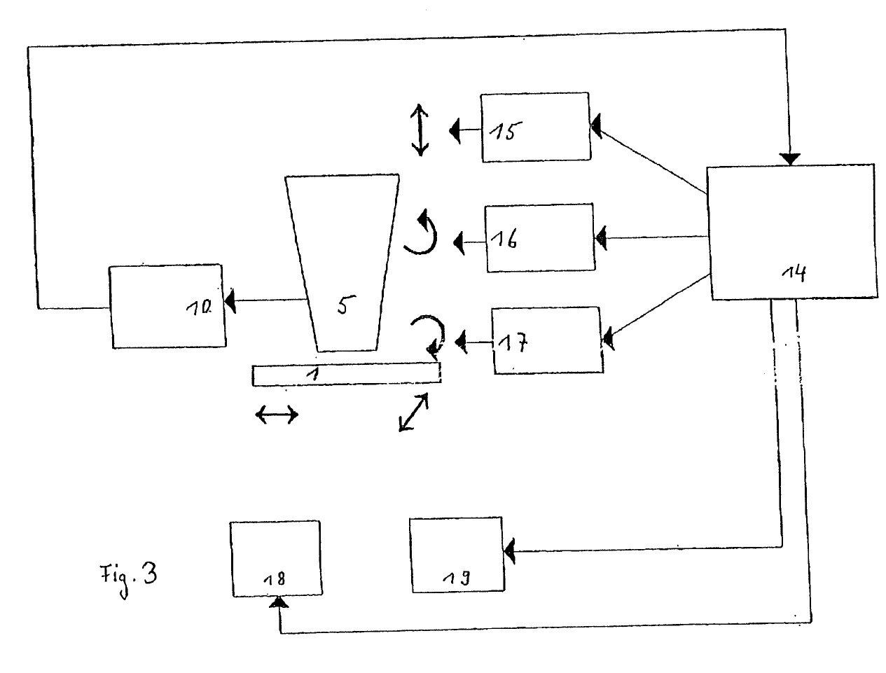

FIG. 3 is a functional block diagram of the device, and

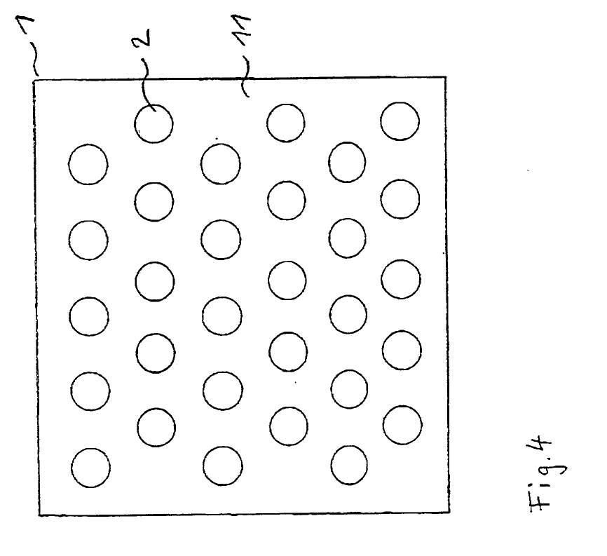

FIG. 4 is a view of the loaded support plane.

FIG. 1 shows schematically the method according to the invention in four steps. Shaped bodies, micro- and/or nano-objects, in the form of polystyren balls with a diameter of 10 micrometers and tubes 4 made from glass and with an internal diameter at its outlet opening 7 of 16 micrometers have been used here. The tubes 4 expand to a diameter of 5 mm at the inlet opening 8.

Respectively 19 tubes 4 are jointed in a hexagon pattern by means of a binding means 20 (Kommentar des Übersetzers: das Bezugszeichen 20 ist nicht in den Zeichnungen enthalten) to a positioning cell 3. The cascading of several positioning cells 3 again in a hexagonal arrangement makes a positioning head 5.

Distance pieces 6 with a length of 12 micrometers are arranged in an outlet plane 9 between the tubes 4, for keeping the distance between the outlet plane 9 of the positioning head 5 and a support plane 11 of the support. The positioning head 5 is moveable via an actuator 15 in the vertical direction. Actuators 16 and 17 serve for moving the positioning head 5 in the x- or y-direction (FIG. 3).The positioning head 5 is elastically suspended in the three axes ( in the direction of the z-axis as well as rotatable around the x- and y-axis). Because of the elasticity in the z-direction the positioning head 5 can be non-destructively placed directly on the support 1, whereby the distance piece 6 guarantees the desired distance between the support plane 11 and the outlet plane 9. The elastic support around the x- and y-axis leads to an automatic compensation of angular errors between the outlet plane 9 and the support plane 11.

A wafer of around 1 cm²made from glass-clear polystyren is used as the support 1, which is provided on the support plane 11 with a few nanometer thick photopolymer layer 12. FIG. 1 shows the support 1 without depressions. Therefore the necessity of a positioning in the x- and y-direction in the range of micrometers is not applicable. A positioning accuracy of few 10 . . . 100 micrometers is sufficient.

After the positioning of the support 1 by means of additional actuators 18 and 19 below the positioning head 5 its downward movement takes place until the distance piece 6 is placed on the support 1. A small excess pressure, which leads to the exiting and placing of the shaped bodies 2, micro and/or nano-objects, which are here foreseen in the form of balls, on the support plane 11, is now applied on the inlet side of the tube 4, which in advance was filled with the liquid phase and which can additionally be treated with ultrasonic sound. The treatment with ultrasonic sound serves amongst others for the separation of the balls.

A UV-lamp 13, which is directed onto the support 1 (FIG. 1), is now switched on for a short time. The polarisation, which is induced by the UV-light, fixes permanently the balls 2 on the support 1 (FIG. 4). Afterwards the positioning head 5 is again lifted by means of the actuator 15. A ring lamp is used as UV-lamp 13, which is arranged around a camera with a microscope objective. If an additional white light is connected at the side into the support 1, the placing of the distance pieces 6 and the balls 3 can be observed from below and can be used for the process control by means of known methods of the industrial image processing. A control device 14 controls and adjusts the actuators 15, 16, 17, 18 and 19, which are responsible for the movement of the positioning head 5 and of the support 1. The data, which is necessary for it, is determined by the sensors 10 and transmitted to the control device 14.

### Reference Numerals

| | |
|---|---|
| 1 | support |
| 2 | Shaped bodies, balls (micro-and/or nano-objects) |
| 3 | positioning cell |
| 4 | capillary tube |
| 5 | positioning head |
| 6 | distance piece |
| 7 | outlet opening |
| 8 | filling hole |
| 9 | outlet plane |
| 10 | sensors |
| 11 | support plane |
| 12 | photopolymer layer |
| 13 | UV-lamp |
| 14 | control device |
| 15 | actuator |
| 16 | actuator |
| 17 | actuator |
| 18 | adjustment actuator |
| 19 | adjustment actuator |
| 20 | binding means |

1. Method for fixing micro- and/or nano-objects, which are contained in a liquid phase, on a support, characterised in that
liquid phases containing several micro- and/or nano-objects (2) are filled into the wide filling holes (8) of conically narrowing tubes (4) and transported in the direction of a narrow outlet opening (7) of the tubes (4), wherein the shape and size of the narrow outlet openings (7) prevent the passage of more than one object (2),
that the narrow outlet openings (7) of the tubes (4) are positioned three-dimensionally (in directions x, y and z) in relation to a support plane (11) before the objects (2) emerge and that the micro- and/or nano-objects (2) having passed through the outlet opening (7) are physically and/or chemically and/or mechanically fixed on the support (1) in the defined position.

2. Method according to claim 1, characterised in that
the transport of the liquid phase including the solid micro- and/or nano-objects (2) through the tubes (4) takes place by means of an applied pressure difference between the wide filling hole (8) and the narrow outlet opening (7).

3. Method according to one of claims 1 or 2, characterised in that
the exiting as well as the positioning and the fixing of the micro- and/or nano-objects takes generally place simultaneously.

4. Method according to one of claims 1 to 3, characterised in that
the support plane (11) is covered with a reactive layer in advance.

5. Method according to one of claims 1 to 4, characterised in that the fixing of the micro- and/or nano-objects (2) is achieved electrostatically and/or photochemically.

6. Method according to one of claims 1 to 4, characterised in that
the fixing of the micro- and/or nano-objects takes place by mechanical means.

7. Method according to one of claims 1 to 4, characterised in that
the fixing of the micro- and/or nano-objects, after these have been magnetised in advance, takes place by magnetic forces.

8. Method according to one of claims 1 to 7, characterised in that
after fixing the micro- and/or nano-objects (2) on the supports (1), they are covered with a layer of gel.

9. Method according to one of claims 1 to 8, characterised in that
for the prevention of a coagulation of the micro- and/or nano-objects (2) in the liquid phase the micro- and/or nano-objects (2) are charged electrostatically in the same sense and the support plane (11) is charged electrostatically in the opposite sense.

10. Method according to one of claims 1 to 9, characterised in that
the micro- and/or nano-objects (2), which are in the same tube (4) are coated with biological-chemical active substances of one type and that the micro- and/or nano-objects (2), which are in different tubes (4), are coated with at least partly different substances.

11. Method according to one of claims 1 to 10, characterised in that
the simultaneous arrangement of different biological-chemical substances are used for the detection of nucleotide sequences.

12. Method according to one of claims 1 to 11, characterised in that
for the detection of nucleotide sequences a test liquid is applied on the support (1), which is provided with the micro- and/or nano-objects (2), and in that via known chemical reactions a macroscopic or microscopic determinable change of properties of the object surface, especially changes in colour or changes of the fluorescence properties are detected.

13. Method according to one of claims 1 to 12, characterised in that
for the prevention of coagulation and adhesion of the micro- and/or nano-objects in the liquid phase, stabilising means, like tensides, are used.

14. Method according to one of claims 1 to 13, characterised in that
capillaries are used as tubes (4).

15. Method according to one of claims 1 to 14, characterised in that
three-dimensional shaped bodies are used as micro- and/or nano-objects or macro-molecules.

16. Device for the execution of the method according to claims 1 to 15, comprising:
a three-dimensional adjustable positioning head (5), which comprises a bundle-like arrangement of conically narrowing tubes (4), which respectively have a wide filling hole (8) and a narrow outlet opening (7),
a support (1) with a support plane (11), which is arranged parallel to an outlet plane (9) of the tubes (4), and
actuators (15, 16, 17) for positioning the outlet openings (7) above the support plane (11) and adjustment actuators (18, 19) for positioning the support (1).

17. Device according to claim 16, characterised in that
the positioning head (5) consists of several positioning cells (3).

18. Device according to claim 16 or 17, characterised in that
distance pieces (6) are arranged at the outlet plane (9).

19. Device according to one of claims 16 to 18, characterised in that
the tubes (4) are capillaries.
